(19) United States
(12) Patent Application Publication
(10) Pub. No.: US 2003/0072777 A1
(43) Pub. Date: Apr. 17, 2003

(54) Combinatorial anti-acne compositions

(76) Inventors: Daniel H. Maes, Huntington, NY (US); Steven F. Schnittger, Huntington Station, NY (US); Chia Wen Chen, Bronxville, NY (US); Mary Steidl Matsui, Teaneck, NJ (US); Kenneth D. Marenus, Dix Hills, NY (US)

Correspondence Address: KAREN A. LOWNEY, ESQ, ESTEE LAUDER COMPANIES, 125 PINELAWN ROAD, MELVILLE,, NY, 11747, US

(21) Appl. No.: 09/971,498

(22) Filed: Oct. 5, 2001

(51) Int. Cl. A61K 35/78; A61K 31/737; A61K 31/7012; A61K 31/685; A61K 35/84

(52) U.S. Cl. 424/401; 424/195.15; 514/53; 514/54; 424/725; 514/78; 424/729

(57) ABSTRACT

The present invention provides a composition containing an anti-acne effective amount of a combination of five components that work combinatorially to combat the acne condition as a whole. The compositions contain an anti-irritant component, an anti-inflammatory component, an adhesion inhibiting component, an sebum reducing component, and a sclareolide component. Both inflamed and non-inflamed lesions associated with the acne condition are reduced with a combination of the anti-irritant component, the anti-inflammatory component comprising at least a hoelen mushroom extract, the adhesion inhibiting component comprising at least a polysaccharide, the sebum reducing component comprising at least a pygeum extract, and the sclareolide component. The methods of the present invention treating the acne condition in a holistic manner. In addition, therefore, the methods of the present invention also include treating and preventing the irritation and inflammation frequently experienced when applying an acne treatment regimen. By applying to the skin an effective amount of the composition of the present invention the appearance of comedones are prevented. The compositions of the present invention protect against and prevent the damaging effects of the acne condition and the appearance of comedones and pustules associated with the acne condition.

### FIELD OF THE INVENTION

The present invention relates to cosmetic or pharmaceutical compositions for treating acne with specific combinatorial components active against the various aspects of the acne condition. More specifically, the invention relates to substantially non-irritating topical compositions free of benzoyl peroxide for the treatment of acne.

### BACKGROUND OF THE INVENTION

The ravages of the acne condition can range from the minor unsightly presence of comedones and scattered papules or pustules to the major disfiguring draining sinuses, scarring and pitting of the skin. As a disease, acne is characterized by a multifactorial pathogenesis including factors of increased sebum production, follicular keratinization, Propionibacterium acnes (P. acnes) proliferation, and inflammation. In general, acne is an androgen dependent disorder. Endocrinological factors effect the secretory activity of sebaceous glands. For example, 5α-reductase catalyzes testosterone to 5α-dihydrotestosterone (DHT), a large amount of which is found in sebaceous glands. Thus, 5α-reductase is considered to be the principal mediator of local androgenic activity. However, the role of 5α-reductase, and in particular, 5α-reductase inhibitors in being able to treat acne is uncertain. A recent report suggests, based on studies, that 5α-reductase inhibitors may not be valuable in acne therapy. Webster, G. F., “Acne Vulgaris: State of the Science”, Arch Dermatol, vol. 135 (September 1999). Still, one element that is far less disputed, if disputed at all, is the role of sebum in causing acne.

Sebum is one of the major factors contributing to the development of the acne condition. Other contributors include colonization by P. acnes, and abnormal keratinization of the follicular epithelium. P. acnes, the major organism responsible for acne, is a gram-positive microaerophilic diphtheroid and it is dominant in the sebaceous follicles. The nutritional needs of P. acnes are provided by the triglyceride fraction of sebum. The glycerol moiety of sebaceous triglycerides are necessary for nutrition and P. acnes gains access to it by an extracellular lipase. As a result of deesterification, the free fatty acids remain in the sebum and the concentration of free fatty acids, in sebum, is directly proportional to the density of P. acnes population. The most common sign of this activity is the presence of the inflammatory response, the comedonal epithelium.

P. acnes within a comedo produces toxic substances which can attack the follicular epithelium and cause rupturing of the comedo. As found in response to other inflammatory situations, neutrophils congregate around the area of the comedo. Neutrophils contain enzymes which are capable of digesting the follicular epithelium and collagen. However, P. acnes is very resistant to degradation by neutrophils or monocytes. Therefore, other treatments are sought to combat the difficult P. acnes. Retinoids, such as, for example, 13-cis retinoic acid (isotretinoin) cause a reduction in sebum production and cause the glands to shrink by about 90 percent. While isotretinoin-induced dryness leads to the elimination of P. acnes, colonization of other microbes may be enhanced by the dry condition of the skin and irritation may develop. Another treatment regimen uses antiandrogens, such as for example, cyproterone and spironolactone, which either block the enzyme, 5α-reductase, or competes with androgens for receptor-binding sites. However, they are only effective after systemically being administered, and as mentioned above, the effect of 5α-reductase inhibitors is uncertain. They, topically, are not known to exert a beneficial effect on reducing sebum production. Plewig, G. and Kligman, A. M., “Acne and Rosacea”, p. 44 (2d ed. 1993) (hereinafter “Acne and Rosacea”).

A widely used treatment regimen is topically applied benzoyl peroxide. The advantage to using benzoyl peroxide in treating acne is its strong antibacterial activity against P. acnes. Application of 5 percent benzoyl peroxide two times daily for five days has been found to reduce P. acnes population by more than 95 percent. In addition, P. acnes has not been found to become resistant to benzoyl peroxide. However, benzoyl peroxide has little effect on sebum production in acne. Some users of benzoyl peroxide as a topical therapeutic treatment, may also experience skin reactions such as irritation and redness. Finally, benzoyl peroxide is unstable and incompatible with other treatment compounds. Therefore, it is difficult to combine benzoyl peroxide with other actives when treating acne. An anti-acne composition free of benzoyl peroxide is desirable because it can contain many actives to treat the multifactorial sub-conditions associated with acne. Other anti-acne agents include sialyl sugars as described in PCT International Publication No. WO 00/06115, manuka oil as reported in “Coast Manuka Oil” on website http://www.coastbio.co.nz/manuka.htm (Jun. 12, 2001), and Acnacidol Biopeptide Base (Acnacidol 101) as reported in “BioSelect Innovations: Products” on website http://www.bioselectinnovations.com/designer.html (Apr. 17, 2001). However, these individually based treatments are not as effective as the combinatorial anti-acne compositions of the present invention. In addition, known acne topical creams, gels, lotions, and emulsions can cause areas of the skin that are otherwise healthy to become irritated due to exposure to pharmacological acne actives. Accordingly, there is a continued need to develop natural alternative ways of treating acne effectively while minimizing the adverse effects of treatment. Accordingly, because of the multifactorial symptoms of acne, a composition that is substantially nonirritating acne yet provides effective treatment of the acne condition is still not known. The present invention now provides a solution to this problem.

### SUMMARY OF THE INVENTION

The present invention relates to topical combinatorial component containing anti-acne compositions comprising an anti-acne effective amount of 1) an adhesion blocking component comprising at least a polysaccharide, 2) a sebum reducing component comprising at least soft pygeum, 3) an anti-irritating component comprising at least a phytosphingosine, 4) an anti-inflammatory component comprising at least hoelen mushroom extract, and 5) a sclareolide component in a cosmetic or pharmaceutical acceptable vehicle. Surprisingly, the present invention achieves superior results with the specific combination of five natural components as they have been found to significantly reduce the severity of the acne condition with substantially no discomfort or irritation. The penta-combinatorial acne treatment of the present invention eliminates known anti-bacterial compounds which are occasionally found to be too strong for the sensitive skin suffering from acne, and which, further provokes discomfort, redness and inflammation of the acne inflicted skin.

The strategy of combining many anti-acne actives in one composition to treat acne is not new, for example, such a combination is disclosed in U.S. Pat. No. 5,976,565 as a patch. Another combination is described in U.S. Pat. No. 4,428,933, contents of which are herein incorporated by reference, wherein a combination of oats, sulfur powder, zinc gluconate, mustard seed, boric acid powder, brewer's yeast, hydrogen peroxide, isopropyl alcohol, water, methyl p. hydroxybenzoate, and egg yolks are described for treating acne. However, the specific combination of efficacious natural anti-acne components of the present invention have not been previously known.

Because of the complexity of the acne problem none of the individual actives in the combinatorial anti-acne treatment of the present invention is effective against acne alone. In addition, combinations of less than all of the actives used in the present invention fail to achieve the full benefits of the present invention. The combination of natural anti-acne components in the present invention reduces inflamed lesions by greater than about 20 percent after the second week of treatment, greater than about 30 percent after four weeks of treatment, and greater than 40 percent after six weeks of treatment. With respect to non-inflamed lesions, the combinatorial anti-acne treatment of the present invention results in a reduction by greater than about 10 percent after the second week of treatment, greater than about 15 percent after four weeks of treatment, and greater than about 20 percent after six weeks of treatment. All results in reducing lesions of any type are accompanied by a substantial lack of discomfort or irritation. These results heretofore have not been seen with the individual natural actives themselves or with smaller combinations of the individual actives of the present invention.

The anti-acne effect achieved by the present compositions, with the use of a natural anti-acne agent, is mild and substantially non-irritating. Consumers feel better about using products that contain natural ingredients because they perceive those ingredients as being milder, safer and healthier. In addition, the network of anti-acne activities is derived from specific sclareolide, adhesion blocking, sebum reducing, anti-inflammatory and anti-irritant components. Thus, the present invention also relates to a method of preventing or protecting the skin against the damaging effects of acne and the appearance of comedones on the skin, which comprises applying to the skin the sclareolide, adhesion blocking component, the sebum reducing component, and the anti-irritant and anti-inflammatory components in anti-acne effective amounts. Thus, the compositions of the present invention provide a natural treatment for the acne condition.

### DETAILED DESCRIPTION OF THE INVENTION

It has been surprisingly found that the combination of five components, namely, the sclareolide component, the adhesion blocking component, the sebum reducing component, the anti-inflammatory component, and the anti-irritant component are effective in treating the acne condition without the use of benzoyl peroxide or salicylic acid. In particular, the five components of the present invention in combination with each other reduce the severity of the acne condition by combating a variety of aspects associated with acne. The primary activity of the present compositions, while not wishing to be bound to any particular theory, is believed to be that a polysaccharide, when applied topically to the skin, inhibits the adhesion of Propionibacterium acnes (P. acnes) to the infundibulum, the region above insertion where the sebaceous gland deposits its contents into the follicular canal via a short duct. Therefore, the P. acnes responsible for the unsightly and damaging symptoms of acne is not able to survive, and its damaging effects to the skin are diminished or eliminated.

The terminal follicles contain the infundibulum, and provide the follicular canal where sebaceous glands empty their contents. Epithelium lines the infundibulum and produces a sturdy, well-differentiated horny layer of cells similar to the epidermis. Further, like the epidermis, the horny layer of the infundibulum has barrier function. It is believed that the development of acne occurs entirely in the sebaceous follicles. Acne and Rosacea, p. 5. The present invention surprisingly has found that the acne condition can be treated on a holistic basis by topical application of polysaccharide in combination with the sclareolide component, the sebum reducing component of at least pygeum extract, the anti-inflammatory component of at least the hoelen mushroom extract, and the anti-irritation component can effectively enhance the treatment of the acne condition.

Although many polysaccharides may be used in the present invention, preferably, the polysaccharide is a sulfated polysaccharide derived from red microalgae of the species, Porphyridium. The polysaccharide is commercially available from Earth Salts Company. As described in U.S. Pat. No. 5,089,481 and International Patent Application WO 97/00689, the red algae polysaccharide is chemically composed of mostly xylose, glucose, and galactose. Both of these references are incorporated herein by reference. In particular, the red algae polysaccharide contains, in addition to galactose, a dimethyl galactose. The red algae polysaccharide contains, in addition to galactose, a dimethyl galactose.

As the algae grows in a liquid medium, polysaccharide is released from the cell surface. Thus, polysaccharide can be collected from the excretions of algae in a growth medium or, alternatively, it can be obtained from the cell walls by extraction. It has been known that algal polysaccharides inhibit the activity of viruses such as human immunodeficiency virus reverse transcriptase enzyme and herpes simplex virus, as described in U.S. Pat. No. 5,089,481 and International Patent Application WO 97/00689. Both of these references are incorporated herein by reference. An acidic polysaccharide antigen has been extracted from the cell walls of P. acnes. Iversen, O-J, et al., “Isolation of an Acidic Polysaccharide Antigen from Propionibacterium acnes”, Arch Dermatol Res, vol. 277, pp. 225-229 (1985). However, the polysaccharide antigen is compositionally different than the red algae polysaccharide preferably used in the present invention. Therefore, it is surprising to find that these polysaccharides are able to reduce the severity of the acne condition when in combination with the other components of the present invention. It has not been known to incorporate the red microalgae polysaccharide of the present invention in a formulation for a cosmetic or pharmaceutical combinatorial anti-acne composition.

To achieve the anti-acne effect in a topical composition, the polysaccharide is present in an anti-acne effective amount. As used herein, an anti-acne effective amount is an amount of polysaccharide sufficient to reduce inflamed or non-inflamed lesions caused by or attributable to P. acnes by an amount comparable to or better than the reduction observed using benzoyl peroxide. Preferably, the polysaccharide is present in an amount of from about 0.05 to about 10%, more preferably from about 0.1 to about 5%, most preferably about 0.5 to about 2%, all by weight of the total composition. The polysaccharide in combination with only the anti-inflammatory component and the anti-irritant component is less effective in treating the lesions, both inflamed and non-inflamed, associated with acne than the polysaccharide in combination with the other four components of the present invention. The red microalgae polysaccharide can be included in any type of cosmetically or pharmaceutically acceptable vehicle for topical application with which it is compatible, e.g., a gel, a cream, a lotion, an ointment, a mousse, a spray, a solid stick, a powder, a suspension, a dispersion, and the like. The polysaccharide can also be provided in a liposome formulation. Techniques for formulating various types of vehicles are well known to those skilled in the art.

In addition to the adhesion blocking component, the compositions of the present invention also include a sebum reducing component which can be any compound known to have sebum reducing activity. Preferably, the sebum reducing component is pygeum extract which is generally known for treating prostate cancer. Extracts of Pygeum africanum have been described by Curri, S. B., et al., in “The Lipid-Sterol Fraction of Pygeum Africanum in Cosmetics”, Chim. Oggi (1), 17-19 (1983), and by Pierini, N., et al., in “Identification and Determination of 1-Docosanol in Extracts of Pygeum Africanum bark and in Pharmaceuticals Containing the Extract”, Boll. Chim. Farm., 121(1), 27-34 (1982). In addition, U.S. Pat. No. 5,972,345, incorporated herein by reference, describes a formulation for treating male pattern hair loss containing African pygeum extract in combination with saw palmetto extract and stinging nettle extract. However, its use in combination with the other components of the present invention, and the beneficial results in treating acne are not known. The pygeum extract is present in an anti-acne effective amount, and the definition of this term as it is described above with respect to the polysaccharide applies to pygeum extract. The pygeum extract is available commercially, as Soft Pygeum Extract, from Actives International, Norwood, N.J. (Alchem International Ltd., Ballabgarh, India). Preferably, the amount of pygeum extract used in combination with the polysaccharide is about 0.05 to about 5.0 percent, more preferably about 0.2 to 2.0 percent, and most preferably about 0.2 to 1.0 percent, all by weight of the composition.

Another preferred compound used as the sebum reducing component is Isolutrol™ (tradename for scymnol sulfate) which can be used alone or in combination with the pygeum extract. Preferably, the sebum reducing component is a combination of isolutrol and pygeum extract. An additional advantage may be experienced with isolutrol because scymnol, a shark bile steroid, according to a research project group, is believed to have dermatological cleansing properties which are considered to be anti-acne in nature. Organic Synthesis Group 1996 Projects, RMIT University, Department of Applied Chemistry, Melbourne, Australia. However, the specific activity in treating acne is not known, and the ability to treat the whole acne condition in combination with the other components of the present invention are not known. The scymnol sulfate is present in an amount of about 0.001 to about 0.05 percent by weight of the present invention. The sebum reducing component can also be a combination of sebum reducing actives and is present in an amount of about 0.02 to about 2.0 when it contains a combination of actives for reducing sebum.

Because acne is inherently an irritating condition associated with inflammation, two requisite components of the present invention are the anti-inflammatory component and the anti-irritant component. Many of the comedones, papules and pustules experienced with acne are inflamed, or become inflamed, as an inflammatory response to the acne condition. Thus, the formulation for the present anti-acne composition, contains an anti-inflammatory component. The anti-inflammatory component can include topical agents, such as, for example, non-steroidal anti-inflammatory drugs, and naturally derived anti-inflammatory agents including but not limited to hoelen mushroom, manuka oil, emu oil, echinacea, chamomile (matricaria oil), soybean protein, calendula, cayenne, tumeric, white willow, sialyl sugars (e.g., 3′ sialyl lactose) and the like. Preferably, the anti-inflammatory component is naturally derived. As used in the present specification and claims, naturally derived agents are those found in nature in animals or plants where natural plant derived agents are referred to as botanicals. While the anti-inflammatory component can include known anti-inflammatory agents, a particular beneficial result in treating acne lesions is found using a hoelen mushroom extract in combination with the other components of the present invention. Hoelen mushroom, or Poria cocos, is an herb used in traditional Chinese and Japanese medicine, and is known as a diuretic, antiviral agent, sedative, fever reducer, and spleen/kidney tonic. An organic or hydro-organic extract of Poria cocos is used in an anti-acne composition in U.S. Pat. No. 5,716,800; however, the present invention incorporating Poria cocos in combination with the four other components of the present invention is surprisingly more effective against acne lesions, both inflamed and non-inflamed, than Poria cocos with only two of the other components of the present invention. The Hoelen mushroom is available from Premier Specialties, Middlesex, N.J. The hoelen mushroom is present in an amount of about 2.0 percent or less, and preferably about 1.0 percent or less.

The anti-irritant component are those which are capable of minimizing the irritation (i.e., responses that are not primarily an inflammatory response) experienced with anti-acne treatment. Examples of irritation include, but are not limited to, itching, redness, flakiness, pain, and the like. Suitable known anti-irritants that can be utilized in the present invention include, but are not limited to, for example, sucrose, green tea extract, hinokitiol, polysaccharide, phytosphingosine, gorgonian extract, sialyl sugars and combinations thereof. The anti-irritant component is present in an amount of about 0.1 to about 5.0 percent by weight of the composition. The phytosphingosine component is known as an added active ingredient in cosmetic and pharmaceutical compositions, as explained in WO 00/01839 and in WO 99/29293, for their anti-inflammatory and antimicrobial activity, and as described in JP 2000109409, for its use in preventing acne comedones. However, WO 00/01839 describes an enhanced method of producing sphingoid bases and derivatives such as phytosphingosines and, WO 99/29293 teaches a combination of a ceramide and a free sphingoid base which, when topically applied, allegedly benefit bacterial, fungal, yeast and viral infections. Therefore, the benefit of phytosphingosine, per se, as a component in a combinatorial anti-acne composition for treating inflamed and non-inflamed lesions has not heretofore been known, especially with respect to irritation. In a preferred embodiment of the present invention, the anti-irritant component is a combination of about 0.1 to about 0.5 percent phytosphingosine, about 0.05 to about 0.2 sialyl sugar, and about 0.2 to about 1.0 percent sucrose.

The compositions of the present invention also contain the sclareolide component. Its use is described in U.S. application Ser. No. 09/773,351 in combination with pygeum extract and amino sugars. It has been reported in U.S. Pat. No. 6,150,381 that sclareol-like and sclareolide-like compounds are useful in treating microbial infections. Sclareol is an important bioactive diterpene obtained from clary sage (Salvia sclarea Labiatae.) The clary sage extract is believed to contain about 70 percent sclareol. In addition, another useful species of the genus Salvia, is Salvia officinalis L. Methods of using Salvia officinalis in an external ointment have been disclosed in U.S. Pat. No. 5,660,831 for controlling high blood-pressure, circulatory problems, and incomplete cicatrization of wounds. The characteristic constituents of Salvia officinalis (Dalmation sage) are believed to be alpha- (about 30 to 40 percent) and beta-thujone (about 10 percent). As used in the present invention, the source of sclareolide can be derived (extracted) naturally from either species of the Salvia genus, or can be synthetically obtained as substantially pure sclareolide. As used in the present specification and claims, substantially pure sclareolide contains greater than 70 percent sclareolide. In the composition, sclareolide is effective in an amount of about 0.01 to about 2.0 percent by weight of the total composition. It is believed to function as an effective desquamation agent when treating acne and in combination with the other components of the present invention.

The compositions of the invention are applied to the skin in a manner appropriate to achieve the intended end result of reducing or eliminating inflamed and non-inflamed lesions associated with the general acne condition as a whole. For example, for the general anti-acne effect, the best results are achieved after regular application over a period of time until the signs of the acne condition cease to persist. It is suggested as an example that topical application of the composition, in an amount of from about 0.1 mg/cm² to 2 mg/cm² of skin, be performed from about once per week to about 4 or 5 times daily, preferably from about 3 times a week to about 3 times daily, most preferably about once or twice per day. The period of topical application may be for a period of at least about two weeks, more preferably from about two weeks to about two years, more preferably from about two weeks to about two months, more preferably still from about two weeks to about six weeks, thereby resulting in the treatment or prevention of the external signs of the acne condition. However, the period of time which may be necessary to treat individual acne conditions will vary, and therefore, repeat applications may ultimately be required. The present invention has the added benefit of being substantially non-irritating, and therefore, treatment can endure as long as necessary to diminish the acne condition without discontinuing its use because of discomfort and the development of irritation.

The methods of the present invention are for treating the acne condition and specifically, treating or preventing the adhesion of P. acnes to keratinocytic cells on the skin. In addition, the present invention prevents and protects the skin against the appearance of unattractive, and even in some cases, disfiguring effects caused by the acne condition by topically applying the compositions of the present invention to the skin. The compositions can be applied to the entire facial area to treat acne and any area of skin on the body which is afflicted with the acne condition, as for example, the back, without causing substantial irritation on the healthy and/or normal areas of the skin surrounding the plagued areas. The method treating the acne condition is achieved by topically applying the sclareolide component, the adhesion blocking component, the sebum reducing component, along with the anti-inflammatory component and the anti-irritant component.

The invention is further illustrated by the following non-limiting examples:

### EXAMPLE I

The following is a composition according to the present invention:

Anti-Acne Composition

| Material | Weight % |
|---|---|
| Phase I | |
| Dimethicone | 0.5 |
| Cetyl Alcohol | 1.0 |
| Glyceryl Stearate | 8.0 |
| Coco-Caprylate | 5.0 |
| Polysorbate 40 | 0.5 |
| Dioctyl Adipate | 5.0 |
| Beeswax | 2.0 |
| Phytosphingosine | 0.2 |
| Sclareolide | 0.1 |
| Manuka oil | 0.1 |
| Phase II | |
| Water | 65.0 |
| Caprylyl Glycol | 1.5 |
| Trisodium EDTA | 0.4 |
| Sucrose | 0.5 |
| Isolutrol | 0.0 |
| Sialyl Sugar | 0.1 |
| Butylene Glycol | 5.0 |
| Glucose Oxidase | 0.1 |
| Glucose | 2.0 |
| Hoelen Mushroom Extract | 0.5 |
| Phase IV | |
| Polysaccharide | 1.0 |
| Pygeum Extract | 0.5 |
| Acnacidol | 1.0 |

### EXAMPLE II

Effect on Inflamed and Non-Inflamed Acne Lesions

A blinded study is conducted to demonstrate the effect of a combination of a polysaccharide and other natural actives on inflamed lesions. as an indicator of its effect as an anti-acne composition. In addition, the same test is conducted to observe the effect on non-inflamed lesions. The study compares the anti-acne compositions of the present invention with the benzoyl peroxide containing compositions. Twenty-five male and female subjects, 18 to 38 years of age, were enrolled to participate in this study. The participants An initial baseline measurement of inflamed and non-inflamed lesions is made before the compositions of the present invention as described in Example I are topically applied to affected areas of the skin. At two, four and six weeks, results are measured by physical observation.

The percent reduction of inflamed and non-inflamed lesions is presented in Tables 1 and 2, below. Data for 5.0% Benzoyl peroxide is derived from Chalker, D. K., M. D., et al., “A Double-blind Study of the Effectiveness of a 3% Erythromycin and 5% Benzoyl Peroxide Combination in the Treatment of Acne Vulgaris”, vol. 9, no. 6, pp. 933-36 (1983), incorporated herein by reference, wherein percent reduction in mean inflammatory lesion counts vs. weeks of therapy is provided in FIG. 4 (numerical data provided in Table 1 below), and percent reduction in mean comedone counts vs. weeks of therapy is provided in FIG. 1 (numerical data provided in Table 2 below).

TABLE 1
AVERAGE PERCENT REDUCTION OF INFLAMED LESIONS

| Treatment | Week 2 | Week 4 | Week 6 |
|---|---|---|---|
| Anti-acne compositions of the | 22 | 40 | 53 |
| present invention | | | |
| 5.0% Benzoyl peroxide | 27 | 21 | 46 |

TABLE 2
AVERAGE PERCENT REDUCTION OF NON-INFLAMED LESIONS

| Treatment | Week 2 | Week 4 | Week 6 |
|---|---|---|---|
| Anti-acne compositions of the | 18 | 31 | 56 |
| present invention | | | |
| 5.0% Benzoyl peroxide | 12 | 15 | 28 |

The lesions, inflamed and non-inflamed, as measured by physical observation, indicate that the skin treated with the compositions of the present invention results in a reduction of both types of lesions. In particular, there is a 22% decrease in inflamed lesions relative to baseline after two weeks, and a 52% decrease after 6 weeks. With respect to non-inflamed lesions, a decrease in 18% of the lesions is found at two weeks, and at 6 weeks there is a 56% decrease. The decrease in inflamed lesions is believed to be due to the effect the combination of polysaccharide and pygeum extract has on P. acnes. These data show that the combination of the anti-irritant component, hoelen mushroom, polysaccharide and pygeum extract results in a reduction in inflamed and non-inflamed lesions, comparable to or better than that of the benzoyl peroxide containing compositions. No subjective discomfort was reported during the course of the study.

### EXAMPLE III

In a study of individual components of the present invention, 45 male and female participants, 18 to 37 years of age, are equally divided into 3 panels. Thus, there are 15 participants on each panel and each panel is assigned one of 3 types of acne treatment creams (I. the first type is the present invention containing an adhesion blocking component of polysaccharide, a sebum reducing component comprising pygeum extract, Acnacidol-p™, and scymnol sulfate, an anti-inflammatory component comprising hoelen mushroom extract, and manuka oil, an anti-irritant component comprising sucrose, sialyl sugar, phytosphingosine, and a sclareolide component, II. the second type is a 3 component cream containing polysaccharide, hoelen mushroom extract, and sucrose, and III. the third type is a 2 component cream containing Acnacidol-p™ and phytosphingosine.) Each acne treatment cream is applied to the full face twice daily, once in the morning and once in the evening for 6 weeks. Participants are instructed not to use any other topical or systemic acne treatment product during the course of the study. Compositions containing polysaccharide in an amount of 1.0 percent, hoelen mushroom extract in an amount of 0.5 percent, and anti-irritation component in an amount of 0.5 percent without the sebum reducing component (3 components) are less effective against the acne condition than the present invention. And, compositions containing an anti-irritant in an amount of 0.2 percent, and a sebum reducing component in an amount of 1.0 percent without polysaccharide and without the anti-inflammatory component (2 components) are less effective against the acne condition than the compositions of the present invention. Results are shown in Table 3 below and demonstrate that the components of the present invention are not cumulative.

TABLE 3
AVERAGE PERCENT REDUCTION OF NON-INFLAMED AND
INFLAMED LESIONS

| Treatment | Week 2 | Week 4 | Week 6 |
|---|---|---|---|
| Anti-acne compositions of the | 36.6 | 59.5 | 87.3 |
| present invention (5 components) | | | |
| 3 components | 21.0 | 40.7 | 53.1 |
| 2 components | 19.4 | 35.0 | 44.0 |

What is claimed is:

1. A topical combinatorial composition comprising an anti-acne effective amount of an adhesion blocking component comprising at least a polysaccharide, a sebum reducing component comprising at least a pygeum extract, an anti-inflammatory component comprising at least a hoelen mushroom extract, an anti-irritant component, and a sclareolide component in a cosmetic or pharmaceutically acceptable vehicle.

2. The composition of claim 1 wherein said polysaccharide is present in an amount of about 0.05 to about 10.0 percent, said pygeum extract is present in an amount of about 0.05 to about 5.0 percent, said hoelen mushroom extract is present in an amount of about 0.01 to about 2.0 percent, the anti-irritant component is present in an amount of about 0.1 to 5.0 percent, and said sclareolide is present in an amount of about 0.01 to 2.0 percent.

3. The composition of claim 1 wherein said anti-irritant is selected from the group consisting of a sucrose, a phytosphingosine, green tea extract, hinokitiol, gorgonian extract, sialyl sugar and combinations thereof.

4. The composition of claim 3 wherein said anti-irritant component comprises about 0.2 to 1.0 percent sucrose, about 0.05 to about 0.2 percent sialyl sugar, and about 0.1 to 0.5 percent phytosphingosine.

5. A benzoyl peroxide-free composition comprising an anti-acne effective amount of an adhesion blocking component comprising at least a polysaccharide, a sclareolide, an anti-inflammatory component comprising at least hoelen mushroom extract, an anti-irritant component, and a sebum reducing component comprising at least a combination of a pygeum extract and a scymnol sulfate.

6. The composition of claim 5 in which said anti-irritant component is a combination of a sucrose, sialyl sugar and a phytosphingosine.

7. The composition of claim 5 in which said polysaccharide is present in an amount of about 0.05 to about 10.0 percent, said scymnol sulfate is present in an amount of about 0.001 to about 0.05 percent, said hoelen mushroom extract is present in an amount of about 0.01 to about 2.0 percent, and the anti-irritant component comprises sucrose in an amount of about 0.2 to 1.0 percent and 0.1 to 0.5 percent phytosphingosine.

8. A method of treating an acne condition on the skin comprising applying to the skin a composition comprising an effective amount of a sclareolide component, a sebum reducing component comprising at least a pygeum extract, an adhesion blocking component comprising at least a polysaccharide, an anti-irritant component comprising at least a sucrose, a sialyl sugar, and a phytosphingosine, and an anti-inflammatory component comprising at least a hoelen mushroom extract.

9. A method of reducing irritation and inflammation on the skin treated for acne comprising topically applying to the skin the composition of claim 1.

10. A method of preventing the appearance of comedones on the skin associated with the acne condition which comprises topically applying to the skin the composition of claim 1.
